SEQUENCE LISTING (4 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Artificial
  Blap mutant
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGPGLDIVAPGVNIQSTYPGSTYASLNGTSMATPHVAGAAALVKQKNPSWSNVQIRNHLKNTATSLGSTNLYGSGLVNAEAATR

SEQ ID NO: 3 - protein, 269 aa, Artificial
  Blap mutant
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 4 - protein, 269 aa, Artificial
  Blap mutant
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2016/0237418 A1
(43) Pub. Date: Aug. 18, 2016

(54) PROTEASE VARIANTS WITH INCREASED STABILITY

(71) Applicant: HENKEL AG & C0. KGAA, Düsseldorf (DE)

(72) Inventors: Hendrik Hellmuth, Duesseldorf (DE); Thomas Weber, Dormagen (DE); Timothy O'Connell, Duesseldorf (DE)

(73) Assignee: Henkel AG & Co. KGaA, Dusseldorf (DE)

(21) Appl. No.: 15/025,597

(22) PCT Filed: Oct. 8, 2014
(86) PCT No.: PCT/EP2014/071529

§ 371 Date: Mar. 29, 2016

(30) Foreign Application Priority Data
Oct. 18, 2013 (DE) 10 2013 221 206.2

(51) Int. Cl. C12N 9/54; C11D 3/386

(52) U.S. Cl. CPC C12N 9/54; C11D 3/386; C12Y 304/21062

(57) ABSTRACT

The invention relates to proteases comprising an amino acid sequence which has an at least 70% sequence identity with the amino acid sequence set forth in SEQ ID NO:1, over the entire length thereof, and in which the amino acids at positions corresponding to positions 3, 4, 99, 188, and 199 according to SEQ ID NO:1 are substituted to S3T, V4I, R99D/E, A188P and V199I, preferably S3T, V4I, R99E, A188P, and V199I, and to the production and use thereof. Such proteases exhibit very good stability, in particular temperature stability, while at the same time having good cleaning power.

### CROSS-REFERENCE TO RELATED APPLICATIONS

This application is a U.S. National-Stage entry under 35 U.S.C. §371 based on International Application No. PCT/EP2014/071529, filed Oct. 8, 2014 which was published under PCT Article 21(2) and which claims priority to German Application No. 10 2013 221 206.2, filed Oct. 18, 2013, which are all hereby incorporated in their entirety by reference.

### TECHNICAL FIELD

The invention falls within the field of enzyme technology. The invention relates to particular proteases and to the production thereof, the amino acid sequence of which has been modified particularly in regard to use in detergents and cleaning agents, to all sufficiently similar proteases having a corresponding modification, and to nucleic acids coding for them. The invention relates further to methods and applications of said proteases and to agents containing them, particularly detergents and cleaning agents.

### BACKGROUND

Proteases are among the technically most important of all enzymes. In the case of detergents and cleaning agents, they are the longest established enzymes present in virtually all modern, high-performance detergents and cleaning agents. They break down protein-containing stains on the items to be cleaned. Among these in turn, subtilisin-like proteases (subtilases, subtilopeptidases, EC 3.4.21.62), which are classed as serine proteases because of the catalytically active amino acids, are particularly important. They act as nonspecific endopeptidases and hydrolyze any acid-amide bonds located within peptides or proteins. Their pH optimum is usually in the clearly alkaline range. An overview of this family can be found, for example, in the article “Subtilases: subtilisin-like proteases” by R. Siezen, pages 75-95, in “Subtilisin Enzymes,” edited by R. Bott and C. Betzel, New York, 1996. Subtilases are formed naturally by microorganisms. Among these, the subtilisins formed and secreted by Bacillus species are to be mentioned in particular as the most significant group within the subtilases.

Examples of subtilisin-like proteases used with preference in detergents and cleaning agents are the subtilisins BPN' and Carlsberg, protease PB92, subtilisins 147 and 309, the protease from Bacillus lentus, in particular from Bacillus lentus DSM 5483, subtilisin DY, and the enzymes, which are to be classified as subtilases but no longer as subtilisins in the strict sense, namely, thermitase, proteinase K, and the proteases TW3 and TW7, as well as variants of the aforesaid proteases that have an amino acid sequence modified as compared with the original protease. Proteases are modified selectively or randomly by methods known from the prior art, and are thus optimized, for example, for use in detergents and cleaning agents. These include point mutagenesis, deletion or insertion mutagenesis, or fusion with other proteins or protein components. Correspondingly optimized variants are thus known for most proteases known from the prior art.

The international patent applications WO 95/23221 and WO 92/21760 disclose variants of the alkaline protease from Bacillus lentus DSM 5483, which are suitable for the use thereof in detergents or cleaning agents. Further, the international patent application WO 2011/032988 discloses detergents and cleaning agents, which likewise contain variants of the alkaline protease from Bacillus lentus DSM 5483. The protease variants disclosed in said publications can be modified apart from other positions at positions 3, 4, 99, and 199 in the enumeration method for alkaline protease from Bacillus lentus DSM 5483 and, for example, have the amino acids 3T, 4I, 99D, 99E, or 1991 at said positions. Modifications, as they are described hereafter, do not emerge from these publications, however.

### DETAILED DESCRIPTION

It has now been found, surprisingly, that a protease of the type of the alkaline protease from Bacillus lentus DSM 5483 or a protease sufficiently similar hereto (based on the sequence identity), which has, in addition to the substitutions 3T, 41, (99D or 99E), and 199I, a substitution of the amino acid at position 188 by proline (188P) in the enumeration method for the alkaline protease from Bacillus lentus DSM 5483, is especially suitable for the use thereof in detergents or cleaning agents and is advantageously improved, particularly with respect to stability.

The subject matter of the invention, therefore, in a first aspect is a protease comprising an amino acid sequence, which has an at least 70% sequence identity with the amino acid sequence set forth in SEQ ID NO:1 over the entire length thereof and which has the amino acid substitutions S3T, V4I, R99D/E, A188P, and V199I, preferably S3T, V4I, R99E, A188P, and V199I, in each case based on the numbering according to SEQ ID NO:1. Preferred, in particular, are such proteases that have no modification in comparison with the original protease at the position, corresponding to position 193 in the enumeration method according to SEQ ID NO:1, particularly those having a V (valine) V193 at this position.

A further subject matter of the invention is a method for producing a protease comprising the substitution of the amino acids at positions, corresponding to positions 3, 4, 99, 188, and 199 in SEQ ID NO:1, in an original protease, which has an at least 70% sequence identity with the amino acid sequence set forth in SEQ ID NO:1 over the entire length thereof, in such a way that the protease comprises the amino acids 3T, 4I, 99D/E, 188P, and 199I, preferably 3T, 4I, 99E, 188P, and 199I, at the corresponding positions.

Especially preferred are such proteases that have no modification in comparison with the original protease at the position, corresponding to position 193 in the enumeration method according to SEQ ID NO:1, particularly those having a V (valine) V193 at this position.

A protease within the meaning of the present patent application therefore comprises both the protease as such and a protease produced using a method of the invention. All statements with regard to the protease therefore refer both to the protease as a substance and to the corresponding methods, particularly the protease production methods.

Associated with the proteases of the invention or the production methods for proteases of the invention as further subject matters of the invention are nucleic acids coding for said proteases, non-human host cells containing proteases of the invention or nucleic acids and agents comprising proteases of the invention, particularly detergents and cleaning agents, washing and cleaning methods, and applications defined by means of the proteases of the invention.

The present invention is based on the surprising realization by the inventors that a modification according to the invention of the positions, corresponding to positions 3, 4, 99, 188, and 199 of the alkaline protease from Bacillus lentus DSM 5483 according to SEQ ID NO:1, in a protease, comprising an amino acid sequence at least 70% identical to the amino acid sequence set forth in SEQ ID NO:1, such that the amino acids 3T, 4I, 99D/E, 188P, and 199I, preferably 3T, 4I, 99E, 188P, and 199I, are present at the corresponding positions, brings about an improved stability of said modified protease in detergents and cleaning agents. This is particularly surprising insofar as the stability of known proteases, which have the substitutions 3T, 4I, 99E, and 199I, is improved synergistically by the introduction of the further substitution at position 188; i.e., the mutation 188P acts surprisingly synergistically with the already achieved stabilizing effects.

The proteases of the invention have a particular stability in detergents or cleaning agents, for example, in regard to surfactants and/or bleaching agents and/or to temperature effects, especially to high temperatures, for example, between 50 and 65° C., particularly 60° C., and/or to acidic or alkaline conditions and/or to changes in pH and/or to denaturing or oxidizing agents and/or to proteolytic degradation and/or to a change in the redox conditions. Consequently, performance-improved protease variants are provided by especially preferred embodiments of the invention. Such advantageous embodiments of the proteases of the invention consequently enable improved washing results of protease-sensitive stains in a broad temperature range.

With respect to the aforementioned international patent applications WO 95/23221, WO 92/21760, and WO 2011/032988, the present invention therefore concerns an alternative sequence modification, which leads to the obtainment of an especially stable and therefore high-performance protease variant for detergents or cleaning agents. This is surprising insofar as the position 188 has already been described previously with a stabilizing effect separately or in combination with the substitution V193M; nevertheless, it was not expected that the combination of a mutation at this position with the substitutions, already with a very great stabilizing effect, at positions 3, 4, 99, and 199 would bring about a further significant increase in stability.

A protease of the invention has proteolytic activity; in other words, it is capable of hydrolyzing peptide bonds of a polypeptide or protein, particularly in a detergent or cleaning agent. A protease of the invention therefore is an enzyme that catalyzes the hydrolysis of peptide bonds and is thereby capable of cleaving peptides or proteins. Further, a protease of the invention preferably is a mature protease, i.e., the catalytically active molecule without signal peptide(s) and/or propeptide(s). Unless otherwise stated, the provided sequences also refer to mature enzymes in each case.

In another embodiment of the invention, the protease, particularly the mature protease, comprises an amino acid sequence, which is at least 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, and 98.8% identical to the amino acid sequence set forth in SEQ ID NO:1 over the entire length thereof, and has the amino acids 3T, 4I, 99D/E, 188P, and 199I, preferably 3T, 4I, 99E, 188P, and 199I, at positions, corresponding to positions 3, 4, 99, 188, and 199 in the enumeration according to SEQ ID NO:1. In connection with the present invention, the feature that a protease has the indicated substitutions means that it contains all corresponding amino acids at the corresponding positions; i.e., none of the five positions is mutated further or, for example, is deleted by fragmentation of the protease. Especially preferred are such proteases that have no modification in comparison with the original protease at the position, corresponding to position 193 in the enumeration method according to SEQ ID NO:1, particularly those having a V (valine) V193 at this position.

Such a protease, preferred according to the invention, is set forth in SEQ ID NO:2.

The identity of nucleic acid or amino acid sequences is determined by a sequence comparison. This sequence comparison is based on the customarily used BLAST algorithm, established in the prior art, (cf., for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs,” Nucleic Acids Res., 25, pp. 3389-3402) and carried out basically by assigning similar sequences of nucleotides or amino acids in the nucleic acid or amino acid sequences to one another. A tabular assignment of the relevant positions is called an alignment. Another algorithm available in the prior art is the FASTA algorithm. Sequence comparisons (alignments), particularly multiple sequence comparisons, are compiled using computer programs. For example, the Clustal series (cf., for example, Chenna et al. (2003): “Multiple sequence alignment with the Clustal series of programs.” Nucleic Acid Research 31, 3497-3500), T-Coffee (cf., for example, Notredame et al. (2000): “T-Coffee: A novel method for multiple sequence alignments.” J. Mol. Biol. 302, 205-217), or programs, based on these programs or algorithms, are frequently used. In the present patent application, all sequence comparisons (alignments) were created using the computer program Vector NTI® Suite 10.3 (Invitrogen Corporation, 1600 Faraday Avenue, Carlsbad, Calif., USA) with the predefined default parameters, whose AlignX module for the sequence comparisons is based on ClustalW.

A comparison of this kind also permits a conclusion on the similarity of the compared sequences. This is usually given as a percent identity, i.e., the proportion of identical nucleotides or amino acid residues at the same positions or at positions corresponding to one another in an alignment. The more broadly construed term of homology includes conserved amino acid exchanges in the case of amino acid sequences, therefore amino acids with a similar chemical activity, because they perform mostly similar chemical activities within the protein. The similarity of compared sequences can therefore also be given as a percent homology or percent similarity. Identity and/or homology data can refer to entire polypeptides or genes or only to individual regions. Homologous or identical regions of various nucleic acid or amino acid sequences are therefore defined by matches in the sequences. Such regions often have identical functions. They can be small and comprise only a few nucleotides or amino acids. Such small regions often perform functions essential for the overall activity of the protein. It can be useful, therefore, to relate sequence matches only to individual, optionally small regions. Unless otherwise stated, the identity or homology data in the present application, however, relate to the entire length of the nucleic acid or amino acid sequence given in each case.

In connection with the present invention, the statement that an amino acid position corresponds to a numerically designated position in SEQ ID NO:1 therefore means that the corresponding position is assigned to the numerically designated position in SEQ ID NO:1 in an alignment as defined above.

In another embodiment of the invention, the protease is characterized in that its cleaning performance compared with that of a protease comprising an amino acid sequence that corresponds to the amino acid sequence set forth in SEQ ID NO:1, 3, or 4, is not significantly reduced, i.e., has at least 80% of the reference washing performance. The cleaning performance can be determined in a washing system containing a detergent in a dose between 4.5 and 7.0 grams per liter of washing liquor and the protease, whereby the proteases to be compared are used in the same concentration (based on the active protein) and the cleaning performance is determined in regard to a stain on cotton, particularly in regard to the stain
blood-milk/ink on cotton: product No. C-05 obtainable from CFT (Center For Testmaterials) B.V. Vlaardingen, Netherlands;
chocolate-milk/rust: product No. C-03 obtainable from CFT (Center For Testmaterials) B.V. Vlaardingen, Netherlands;
milk/oil: product No. PC-10 obtainable from CFT (Center For Testmaterials) B.V. Vlaardingen, Netherlands;
whole egg/pigment: product No. 10N WFK 10N (whole egg/pigment on cotton, wfk—Cleaning Technology Institute e.V., Krefeld, Germany); and
cocoa: product No. EMPA 112, EMPA 112 (cocoa on cotton, Eidgenossische Material- and Prüfanstalt (EMPA) Testmaterialien AG [Swiss Federal Laboratories for Materials Science & Technology (EMPA) Test Materials], St. Gallen, Switzerland);
   by measuring the degree of whiteness of the washed textiles, the washing process being performed for 70 minutes at a temperature of 40° C. and the water having a water hardness between 15.5 and 16.5° (German degrees of hardness). The concentration of the protease in the detergent designated for this washing system is 0.001 to 0.1% by weight, preferably of 0.01 to 0.06% by weight, based on active protein.

A preferred liquid detergent for a washing system of this type has the following composition (all quantities given in percent by weight): 0.3 to 0.5% xanthan gum, 0.2 to 0.4% anti-foaming agent, 6 to 7% glycerol, 0.3 to 0.5% ethanol, 4 to 7% FAEOS (fatty alcohol ether sulfate), 24 to 28% nonionic surfactants, 1% boric acid, 1 to 2% sodium citrate (dihydrate), 2 to 4% sodium carbonate, 14 to 16% coconut fatty acids, 0.5% HEDP (1-hydroxyethane-(1,1-diphosphonic acid)), 0 to 0.4% PVP (polyvinylpyrrolidone), 0 to 0.05% optical brightener, 0 to 0.001% dye, remainder demineralized water. Preferably the dose of the liquid detergent is between 4.5 and 6.0 grams per liter of washing liquor, for example, 4.7, 4.9, or 5.9 grams per liter of washing liquor. The washing preferably takes place in a pH value range between pH 8 and pH 10.5, preferably between pH 8 and pH 9.

A preferred powdered detergent for a washing system of this type has the following composition (all quantities given in percent by weight): 10% linear alkylbenzene sulfonate (sodium salt), 1.5% C12-C18 fatty alcohol sulfate (sodium salt), 2.0% C12-C18 fatty alcohol with 7 EO, 20% sodium carbonate, 6.5% sodium hydrogen carbonate, 4.0% amorphous sodium disilicate, 17% sodium carbonate peroxyhydrate, 4.0% TAED, 3.0% polyacrylate, 1.0% carboxymethylcellulose, 1.0% phosphonate, 27% sodium sulfate, remainder: foam inhibitors, optical brightener, fragrances. Preferably, the dose of the powdered detergent is between 4.5 and 7.0 grams per liter of washing liquor, for example, and particularly preferably 4.7 grams per liter of washing liquor, or 5.5, 5.9, or 6.7 grams per liter of washing liquor. The washing preferably takes place in a pH value range between pH 9 and pH 11.

Within the scope of the invention, the cleaning performance is determined at 40° C. with use of a liquid detergent as indicated above, whereby the washing process preferably takes place for 70 minutes.

The degree of whiteness, i.e., the lightening of the stains, is determined as a measure of the cleaning performance preferably using optical measurement methods, preferably photometrically. A device suitable for this is, for example, the Minolta CM508d spectrometer. The devices used for the measurement are usually calibrated beforehand using a white standard, preferably a provided white standard.

Methods for determining protease activity are familiar to the skilled artisan in the field of enzyme technology and are used routinely by him. Such methods are disclosed, for example, in Tenside, Vol. 7 (1970), pp. 125-132. Alternatively, the protease activity can be determined via the release of the chromophore para-nitroaniline (pNA) from the substrate suc-L-Ala-L-Ala-L-Pro-L-Phe-p-nitroanilide (AAPF). The protease cleaves the substrate and releases pNA. The release of pNA causes an increase in extinction at 410 nm, whose time course is a measure of enzymatic activity (cf. Del Mar et al., 1979). The measurement is performed at a temperature of 25° C., pH 8.6, and a wavelength of 410 nm. The measurement time is 5 minutes and the measurement interval 20 seconds to 60 seconds. The protease activity is usually given in protease units (PU). Suitable protease activities are, for example, 2.25, 5, or 10 PU per milliliter of washing liquor. The protease activity is not equal to zero, however.

The protein concentration can be determined with the aid of known methods, for example, the BCA method (bicinchoninic acid; 2,2′-biquinolyl-4,4′-dicarboxylic acid) or the Biuret method (A. G. Gornall, C. S. Bardawill, and M. M. David, J. Biol. Chem., 177 (1948), pp. 751-766). The active protein concentration can be determined in this regard by titrating the active sites with use of a suitable irreversible inhibitor (for proteases, for example, phenylmethylsulfonyl fluoride (PMSF)) and by determining the residual activity (cf. M. Bender et al., J. Am. Chem. Soc. 88, 24 (1966), pp. 5890-5913).

Proteins can be combined into groups of immunologically related proteins by the reaction with an antiserum or a specific antibody. The members of such a group are characterized in that they have the same antigenic determinant recognized by an antibody. They are structurally so similar, therefore, that they are recognized by an antiserum or specific antibodies. A further subject matter of the invention therefore are proteases that are characterized in that they have at least one and more preferably two, three, or four corresponding antigenic determinants with a protease of the invention. Such proteases based on their immunological similarities are structurally so similar to the proteases of the invention that they can also be assumed to have the same function.

In addition to the amino acid modifications described above, the proteases of the invention can have further amino acid modifications, particularly amino acid substitutions, insertions, or deletions. Such proteases are developed further, for example, by targeted genetic modification, i.e., by mutagenesis methods, and optimized for specific purposes or with regard to special properties (for example, with regard to their catalytic activity, stability, etc.). Further, nucleic acids of the invention can be introduced into recombination batches and thereby used to create entirely novel proteases or other polypeptides.

The aim is to introduce targeted mutations such as substitutions, insertions, or deletions into the known molecules in order to improve, for example, the cleaning performance of the enzymes of the invention. To this end, in particular the surface charges and/or the isoelectric point of the molecules and thereby their interactions with the substrate can be modified. Thus, for example, the net charge of the enzymes can be modified in order to influence thereby the substrate binding, particularly for use in detergents and cleaning agents. Alternatively or in addition, the stability of the protease can be increased still further by one or more appropriate mutations and its cleaning performance can be improved as a result. Advantageous properties of individual mutations, e.g., individual substitutions, can complement one another. A protease, already optimized with regard to certain properties, for example, in terms of its stability with respect to surfactants and/or bleaching agents and/or other components, can therefore be developed further within the context of the invention.

The following convention is used to describe substitutions that relate to just one amino acid position (amino acid exchanges): first, the naturally occurring amino acid is identified in the form of the internationally accepted one-letter code; this is followed by the associated sequence position and finally the inserted amino acid. Multiple exchanges within the same polypeptide chain are separated from one another by means of slashes. In the case of insertions, additional amino acids are listed after the sequence position. In the case of deletions, the missing amino acid is replaced by a symbol such as an asterisk or a dash or a Δ is provided before the corresponding position. For example, A95G describes the substitution of alanine at position 95 with glycine, A95AG the insertion of glycine after the amino acid alanine at position 95, and A95* or ΔA95 the deletion of alanine at position 95. This nomenclature is familiar to the skilled artisan in the field of enzyme technology.

A further subject matter of the invention therefore is a protease, which is characterized in that it is obtainable from a protease as described above as the parent molecule by a single or multiple conservative amino acid substitution, said protease, in the enumeration according to SEQ ID NO:1, still having the amino acid substitutions of the invention at positions corresponding to positions 3, 4, 99, 188, and 199 in SEQ ID NO:1, as described above. The term “conservative amino acid substitution” means the exchange (substitution) of one amino acid residue for another amino acid residue, whereby this exchange does not lead to a change in the polarity or charge at the position of the exchanged amino acid, e.g., the exchange of one nonpolar amino acid residue for another nonpolar amino acid residue. Conservative amino acid substitutions within the scope of the invention comprise, for example: G=A=S, I=V=L=M, D=E, N=Q, K=R, Y=F, S=T, G=A=I=V=L=M=Y=F=W=P=S=T. Especially preferred are such proteases that have no modification in comparison with the original protease at the position, corresponding to position 193 in the enumeration method according to SEQ ID NO:1, particularly those having a V (valine) V193 at this position.

Alternatively or in addition, the protease is characterized in that it is obtainable from a protease of the invention as the parent molecule by fragmentation or by deletion, insertion, or substitution mutagenesis and comprises an amino acid sequence that matches the parent molecule over a length of at least 50, 60, 70, 80, 90, 100, 110, 120, 130, 140, 150, 160, 170, 180, 190, 200, 210, 220, 230, 240, 250, 260, 265, or 266 contiguous amino acids, the mutated amino acid residues in the parent molecule at positions, corresponding to positions 3, 4, 99, 188, and 199 in SEQ ID NO:1, still being present. Especially preferred are such proteases that have no modification in comparison with the original protease at the position, corresponding to position 193 in the enumeration method according to SEQ ID NO:1, particularly those having a V (valine) V193 at this position.

Thus, for example, it is possible to delete individual amino acids at the termini or in the loops of the enzyme, without the proteolytic activity being lost or reduced thereby. Further, for example, the allergenicity as well of the relevant enzymes can be reduced and thus their usability improved overall by such fragmentation and deletion, insertion, or substitution mutagenesis. Advantageously, the enzymes retain their proteolytic activity after mutagenesis as well; i.e., their proteolytic activity corresponds at least to that of the parent enzyme; i.e., in a preferred embodiment, the proteolytic activity constitutes at least 80%, preferably at least 90% of the activity of the parent enzyme. Further substitutions can also have advantageous effects. Both individual and multiple contiguous amino acids can be exchanged for other amino acids.

Alternatively or in addition, the protease is characterized in that it is obtainable from a protease of the invention as the parent molecule by one or more amino acid substitutions at positions assigned in an alignment to positions 36, 42, 47, 56, 61, 69, 87, 96, 101, 102, 104, 114, 118, 120, 130, 139, 141, 142, 154, 157, 193, 205, 211, 224, 229, 236, 237, 242, 243, 255, and 268 of the protease from Bacillus lentus according to SEQ ID NO:1, the protease in the enumeration according to SEQ ID NO:1 still having the substitutions at the positions, corresponding to positions 3, 4, 99, 188, and 199 in SEQ ID NO:1, as described above. The other amino acid positions are hereby defined by an alignment of the amino acid sequence of a protease of the invention with the amino acid sequence of the protease from Bacillus lentus, as they are set forth in SEQ ID NO:1. Furthermore, the assignment of the positions is based on the mature protein. This assignment should also be used particularly if the amino acid sequence of a protease of the invention comprises a higher number of amino acid residues than the protease from Bacillus lentus according to SEQ ID NO:1. Starting from the specified positions in the amino acid sequence of the protease from Bacillus lentus, the modification positions in a protease of the invention are those assigned precisely to said positions in an alignment. Especially preferred are such proteases that have no modification in comparison with the original protease at the position, corresponding to position 193 in the enumeration method according to SEQ ID NO:1, particularly those having a V (valine) V193 at this position.

Advantageous positions for sequence modifications, particularly substitutions, of the protease from Bacillus lentus, which are preferably of importance when transferred to homologous positions of the proteases of the invention and impart advantageous functional properties to the protease, are accordingly the positions 36, 42, 47, 56, 61, 69, 87, 96, 101, 102, 104, 114, 118, 120, 130, 139, 141, 142, 154, 157, 193, 205, 211, 224, 229, 236, 237, 242, 243, 255, and 268, for assignment in an alignment with SEQ ID NO:1 and thereby in the enumeration according to SEQ ID NO:1. The following amino acid residues are located at the specified positions in the wild type molecule of the protease from Bacillus lentus: S36, N42, A47, T56, G61, T69, E87, A96, A101, I102, 5104, N114, H118, Al20, 5130, 5139, T141, S142, S154, S157, V193, G205, L211, A224, K229, 5236, N237, N242, H243, N255, or T268. The mutation M193V can optionally be covered by others. Especially preferred, however, are such proteases that have no modification in comparison with the original protease at the position, corresponding to position 193 in the enumeration method according to SEQ ID NO:1, particularly those having a V (valine) V193 at this position.

In particular, substitutions G61A, S154D, and S154E, for example, are advantageous, unless the corresponding homologous positions in a protease of the invention are already naturally occupied by one of said preferred amino acids.

Further confirmation of the correct assignment of amino acids to be modified, i.e., in particular their functional equivalence, can be provided by comparative experiments, in which the two positions assigned to one another on the basis of an alignment are modified in the same way in both proteases being compared and it is observed whether the enzymatic activity is modified in the same way in both cases. If, for example, an amino acid exchange at a particular position of the protease from Bacillus lentus according to SEQ ID NO:1 is accompanied by a change in an enzyme parameter, for example, by an increase in the K_{M} value, and if a corresponding change in the enzyme parameter, thus, for example, likewise an increase in the K_{M} value, is observed in a protease variant of the invention whose amino acid exchange was achieved by the same inserted amino acid, this can be regarded as a confirmation of the correct assignment.

All of the specified elements can also be applied to the method of the invention for producing a protease. Accordingly, a method of the invention comprises further one or more of the following process steps:
(a) introducing a single or multiple conservative amino acid substitution, whereby the protease in the enumeration according to SEQ ID NO:1 has the amino acid substitutions 3T, 4I, 99D/E, 188P, and 199I, preferably 3T, 4I, 99E, 188P, and 199I;
(b) modifying the amino acid sequence by fragmentation or by deletion, insertion, or substitution mutagenesis such that the protease comprises an amino acid sequence, which matches the parent molecule over a length of at least 50, 60, 70, 80, 90, 100, 110, 120, 130, 140, 150, 160, 170, 180, 190, 200, 210, 220, 230, 240, 250, 260, 265, or 266 contiguous amino acids, the amino acid substitutions 3T, 4I, 99D/E, 188P, and 199I, preferably 3T, 4T, 99E, 188P, and 199I, in the parent molecule still being present;
(c) introducing a single or multiple amino acid substitution into one or more of the positions, assigned in an alignment to positions 36, 42, 47, 56, 61, 69, 87, 96, 101, 102, 104, 114, 118, 120, 130, 139, 141, 142, 154, 157, 193, 205, 211, 224, 229, 236, 237, 242, 243, 255, and 268 of the protease from Bacillus lentus according to SEQ ID NO:1, whereby the protease in the enumeration according to SEQ ID NO:1 has the amino acid substitutions 3T, 4I, 99D/E, 188P, and 199I, preferably 3T, 4I, 99E, 188P, and 1991. Especially preferred are such proteases that have no modification in comparison with the original protease at the position, corresponding to position 193 in the enumeration method according to SEQ ID NO:1, particularly those having a V (valine) V193 at this position.

All statements also apply to the methods of the invention.

In other embodiments of the invention, the protease or the protease produced using the method of the invention is still at least 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, or 98.8% identical to the amino acid sequence set forth in SEQ ID NO:2 over the entire length thereof. Alternatively the protease or the protease produced using the method of the invention is still at least 70%, 71%, 72%, 73%, 74%, 75%, 76%, 77%, 78%, 79%, 80%, 81%, 82%, 83%, 84%, 85%, 86%, 87%, 88%, 89%, 90%, 90,5%, 91%, 91.5%, 92%, 92.5%, 93%, 93.5%, 94%, 94.5%, 95%, 95.5%, 96%, 96.5%, 97%, 97.5%, or 98% identical to the amino acid sequence set forth in SEQ ID NO:1 over the entire length thereof. The protease or the protease produced using a method of the invention has the amino acid substitutions 3T, 4I, 99D/E, 188P, and 199I, preferably 3T, 4I, 99E, 188P, and 1991.

Especially preferred are such proteases that have no modification in comparison with the original protease at the position, corresponding to position 193 in the enumeration method according to SEQ ID NO:1, particularly those having a V (valine) V193 at this position.

A further subject matter of the invention is a previously described protease, which is stabilized in addition, particularly by one or more mutations, for example, substitutions, or by coupling to a polymer. An increase in the stability during storage and/or during use, for example, in the washing process, then has the result that the enzymatic activity lasts longer and the cleaning performance is thereby improved. Basically all expedient stabilization options and/or those described in the prior art may be used. Preferred stabilizations are those that are achieved by mutations of the enzyme itself, because such stabilizations require no further work steps after recovery of the enzyme. Examples of sequence modifications suitable for this purpose are given above. Other suitable sequence modifications are known from the prior art. Thus, for example, proteases can also be stabilized by exchanging one or more tyrosine residues for other amino acids.

Further options for stabilization are, for example:
Modifying the binding of metal ions, in particular of calcium binding sites, for example, by exchanging one or more of the amino acids involved in the calcium binding for one or more negatively charged amino acids and/or by introducing sequence modifications in at least one of the sequences of the two amino acids arginine/glycine;
Protecting from the effect of denaturing agents such as surfactants by mutations that cause a change in the amino acid sequence on or at the surface of the protein;
Exchanging amino acids, located close to the N-terminus, for those that presumably come into contact with the rest of the molecule by means of noncovalent interactions and thus contribute to maintaining the globular structure.

Preferred embodiments are those in which the enzyme is stabilized in multiple ways, because multiple stabilizing mutations act additively or synergistically.

A further subject matter of the invention is a protease as described above, which is characterized in that it has at least one chemical modification. A protease with such a modification is designated as a derivative; i.e., the protease is derivatized.

Derivatives within the meaning of the present application accordingly are understood as proteins whose pure amino acid chain has been chemically modified. Such derivatizations can be performed, for example, in vivo by the host cell expressing the protein. Couplings of low-molecular-weight compounds, such as lipids or oligosaccharides, are to be emphasized in particular in this regard. Derivatizations can also be carried out in vitro, however, for instance, by the chemical conversion of a side chain of an amino acid or by covalent binding of a different compound to the protein. Coupling of amines to carboxyl groups of an enzyme in order to modify the isoelectric point is possible, for example. Another such compound can also be a further protein that is bound, for example, by bifunctional chemical compounds to a protein of the invention. Derivatization is likewise to be understood as covalent binding to a macromolecular carrier, or also as a noncovalent inclusion into suitable macromolecular cage structures. Derivatizations, for example, can influence the substrate specificity or strength of binding to the substrate, or can bring about a temporary blocking of enzymatic activity if the coupled substance is an inhibitor. This can be useful, for example, for the period of storage. Modifications of this kind can furthermore influence stability or enzymatic activity. They can moreover also serve to decrease the allergenicity and/or immunogenicity of the protein and thereby, for example, to increase its skin compatibility. For example, couplings to macromolecular compounds, for example, polyethylene glycol, can improve the protein with regard to stability and/or skin compatibility.

Derivatives of a protein of the invention can also be understood in the broadest sense as preparations of said proteins. Depending on the recovery, processing, or preparation, a protein can be associated with a variety of other substances, for example, from the culture of the producing microorganisms. A protein can also have had other substances deliberately added to it, for example, in order to increase its storage stability. For this reason, all preparations of a protein of the invention are also novel. This is also irrespective of whether or not it actually displays this enzymatic activity in a specific preparation. It may be desirable for it to possess little or no activity during storage and to develop its enzymatic function only at the time of use. This can be controlled, for example, by suitable accompanying substances. In particular, the joint preparation of proteases with protease inhibitors is possible in this regard.

With respect to all proteases or protease variants and/or derivatives described above within the scope of the present invention, those are particularly preferred whose stability and/or activity corresponds at least to those of the protease according to SEQ ID NO:2, and/or whose cleaning performance corresponds at least to that of the protease according to SEQ ID NO:2, whereby the cleaning performance is determined in a washing system as described above.

A further subject matter of the invention is a nucleic acid coding for a protease of the invention, and a vector containing such a nucleic acid, particularly a cloning vector or an expression vector.

These can be DNA or RNA molecules. They can exist as a single strand, as a single strand complementary to said single strand, or as a double strand. In the case of DNA molecules, in particular, the sequences of both complementary strands in all three possible reading frames are to be considered in each case. It must be considered further that different codons, therefore, base triplets, can code for the same amino acids, so that a specific amino acid sequence can be encoded by multiple different nucleic acids. Because of this degeneracy of the genetic code, all nucleic acid sequences that can encode one of the above-described proteases are included in this subject matter of the invention. The skilled artisan is capable of unequivocally determining these nucleic acid sequences, because despite the degeneracy of the genetic code, defined amino acids are to be assigned to individual codons. The skilled artisan, proceeding from an amino acid sequence, can therefore readily ascertain nucleic acids coding for said amino acid sequence. Furthermore, in the case of nucleic acids of the invention one or more codons can be replaced by synonymous codons. This aspect refers in particular to the heterologous expression of the enzymes of the invention. Every organism, for example, a host cell of a production strain, possesses a specific codon usage. Codon usage is understood as the translation of the genetic code into amino acids by the respective organism. Bottlenecks in protein biosynthesis can occur if the codons located on the nucleic acid are faced with a comparatively small number of charged tRNA molecules in the organism. Although coding for the same amino acid, the result is that a codon is translated less efficiently in the organism than a synonymous codon coding for the same amino acid. Because of the presence of a higher number of tRNA molecules for the synonymous codon, the latter can be translated more efficiently in the organism.

Using methods commonly known today such as, for example, chemical synthesis or the polymerase chain reaction (PCR) in combination with standard methods of molecular biology or protein chemistry, a skilled artisan is capable of producing, on the basis of known DNA sequences and/or amino acid sequences, the corresponding nucleic acids up to complete genes. Such methods are known, for example, from Sambrook, J., Fritsch, E. F. and Maniatis, T. 2001. Molecular Cloning: A Laboratory Manual, 3rd Edition, Cold Spring Laboratory Press.

Within the meaning of the present invention, vectors are understood to be elements, made up of nucleic acids and containing a nucleic acid of the invention as a characterizing nucleic acid region. They make it possible to establish said nucleic acid as a stable genetic element in a species or a cell line over multiple generations or cell divisions. When used in bacteria, in particular, vectors are special plasmids, therefore, circular genetic elements. Within the scope of the present invention, a nucleic acid of the invention is cloned into a vector. Vectors include, for example, those originating from bacterial plasmids, viruses, or bacteriophages, or predominantly synthetic vectors or plasmids having elements of very diverse origin. With the further genetic elements present in each case, vectors are capable of establishing themselves as stable units in the relevant host cells over multiple generations. They can be present extrachromosomally as separate units or be integrated into a chromosome or into chromosomal DNA

Expression vectors comprise nucleic acid sequences that enable them to replicate in the host cells containing them, preferably microorganisms, especially preferably bacteria, and to express a nucleic acid contained therein. The expression is influenced in particular by the promoter(s) that regulate transcription. In principle, the expression can be carried out by the natural promoter, originally located before the nucleic acid to be expressed, but also by a host cell promoter provided on the expression vector or by a modified or completely different promoter of a different organism or a different host cell. In the present case, at least one promoter is provided for the expression of a nucleic acid of the invention and used for the expression thereof. Expression vectors can furthermore be regulated, for example, by a change in culturing conditions or when the host cells containing them reach a specific cell density, or by the addition of specific substances, in particular activators of gene expression. One example of such a substance is the galactose derivative isopropyl-β-D-thiogalactopyranoside (IPTG), which is used as an activator of the bacterial lactose operon (lac operon). In contrast to expression vectors, the contained nucleic acid is not expressed in cloning vectors.

A further subject matter of the invention is a non-human host cell that contains a nucleic acid of the invention or a vector of the invention, or that contains a protease of the invention, particularly one that secretes the protease into the medium surrounding the host cell. A nucleic acid of the invention or a vector of the invention is preferably transformed into a microorganism, which then represents a host cell of the invention. Alternatively, individual components, i.e., nucleic acid parts or fragments of a nucleic acid of the invention, can also be introduced into a host cell in such a way that the then resulting host cell contains a nucleic acid of the invention or a vector of the invention. This procedure is especially suitable if the host cell already contains one or more constituents of a nucleic acid of the invention or a vector of the invention, and the further constituents are then added accordingly. Cell transformation methods are established in the prior art and are sufficiently known to the skilled artisan. All cells are suitable in principle as host cells, i.e., prokaryotic or eukaryotic cells. Preferred are host cells that can be advantageously manipulated genetically, for example, as regards the transformation using the nucleic acid or vector and the stable establishment thereof, for example, single-celled fungi or bacteria. Further, preferred host cells are notable for being readily manipulated in microbiological and biotechnological terms. This refers, for example, to easy culturability, high growth rates, low requirements for fermentation media, and good production and secretion rates for foreign proteins. Preferred host cells of the invention secrete the (transgenically) expressed protein into the medium surrounding the host cells. Furthermore, the proteases can be modified after their production by cells producing them, for example, by the addition of sugar molecules, formylations, aminations, etc. Post-translational modifications of this kind can functionally influence the protease.

Further preferred embodiments are represented by those host cells whose activity can be regulated on the basis of genetic regulatory elements that are provided, for example, on the vector, but can also be present at the outset in these cells. They can be stimulated to expression, for example, by the controlled addition of chemical compounds serving as activators, by changing the culturing conditions, or when a specific cell density is reached. This makes possible an economic production of the proteins of the invention. One example of such a compound is IPTG, as described above.

Preferred host cell are prokaryotic or bacterial cells. Bacteria are notable for short generation times and low demands in terms of culturing conditions. As a result, cost-effective culturing methods or production methods can be established. In addition, the skilled artisan has a wide range of experience in the case of bacteria in fermentation technology. Gram-negative or Gram-positive bacteria may be suitable for a specific production, for very different reasons to be determined experimentally in the individual case, such as nutrient sources, product formation rate, time requirement, etc.

In Gram-negative bacteria such as, for example, Escherichia coli, a plurality of proteins are secreted into the periplasmic space, therefore, into the compartment between the two membranes enclosing the cells. This can be advantageous for specific applications. Further, Gram-negative bacteria can also be developed so that they discharge the expressed proteins not only into the periplasmic space but into the medium surrounding the bacterium. Gram-positive bacteria, in contrast, such as, for example, bacilli or actinomycetes, or other representatives of the Actinomycetales, possess no external membrane, so that secreted proteins are released directly into the medium, as a rule the nutrient medium, surrounding the bacteria, from which medium the expressed proteins can be purified.

They can be isolated directly from the medium or processed further. In addition, Gram-positive bacteria are related or identical to most source organisms for technically important enzymes, and usually themselves form comparable enzymes, so that they have a similar codon usage and their protein synthesis apparatus is naturally organized accordingly.

Host cells of the invention can be modified in terms of their requirements for culture conditions, can have other or additional selection markers, or can also express other or additional proteins. They can also be, in particular, host cells that transgenically express multiple proteins or enzymes.

The present invention can be used in principle with all microorganisms, particularly with all fermentable microorganisms, particularly preferably with those of the genus Bacillus, and has the result that the proteins of the invention can be produced with the use of such microorganisms. Such microorganisms then represent host cell within the meaning of the invention.

In a further embodiment of the invention, the host cell is characterized in that it is a bacterium, preferably one that is selected from the group of genera comprising Escherichia, Klebsiella, Bacillus, Staphylococcus, Corynebacterium, Arthrobacter, Streptomyces, Stenotrophomonas, and Pseudomonas, more preferably one that is selected from the group comprising Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor, and Stenotrophomonas maltophilia.

The host cell can also be a eukaryotic cell, however, which is characterized in that it possesses a cell nucleus. A further subject matter of the invention therefore is a host cell characterized in that it has a cell nucleus. Unlike prokaryotic cells, eukaryotic cells are capable of post-translationally modifying the formed protein. Examples thereof are fungi such as actinomycetes or yeasts such as Saccharomyces or Kluyveromyces. This may be especially advantageous, for example, if the proteins are to undergo specific modifications, enabled by such systems, in connection with their synthesis. Modifications that eukaryotic systems carry out particularly in conjunction with protein synthesis include, for example, the binding of low-molecular-weight compounds such as membrane anchors or oligosaccharides. Oligosaccharide modifications of this kind can be desirable, for example, in order to lower the allergenicity of an expressed protein. Coexpression with the enzymes naturally formed by such cells, for example, cellulases or lipases, can also be advantageous. Furthermore, thermophilic fungal expression systems, for example, can be particularly suitable for the expression of temperature-resistant proteins or variants.

The host cells of the invention are cultured and fermented in a conventional manner, for example, in discontinuous or continuous systems. In the former case, a suitable nutrient medium is inoculated with the host cells, and the product is harvested from the medium after a period of time to be determined experimentally. Continuous fermentations are notable for the achievement of a dynamic equilibrium in which, over a comparatively long time period, some cells die off but also regenerate, and the formed protein can be removed simultaneously from the medium.

Host cells of the invention are used preferably to produce proteases of the invention. A further subject matter of the invention therefore is a method for producing a protease comprising
a) culturing a host cell of the invention
b) isolating the protease from the culture medium or from the host cell.

Said subject matter of the invention preferably comprises fermentation methods. Fermentation methods are known per se from the prior art and represent the actual large-scale production step, generally followed by a suitable purification method for the produced product, for example, the protease of the invention. All fermentation methods based on a suitable method for producing a protease of the invention represent embodiments of said subject matter of the invention.

Fermentation methods which are characterized in that fermentation is carried out via a feed strategy are particularly appropriate. In this case, the media constituents consumed during continuous culturing are fed in. Considerable increases both in cell density and in cell mass or dry mass and/or especially in the activity of the protease of interest can be achieved in this way. Further, the fermentation can also be designed so that undesirable metabolic products are filtered out or are neutralized by the addition of buffers or suitable counterions.

The produced protease can be harvested from the fermentation medium. A fermentation method of this kind is preferred over isolation of the protease from the host cell, i.e., product recovery from the cell mass (dry mass), but requires the provision of suitable host cells or one or more suitable secretion markers or mechanisms and/or transport systems, so that the host cells secrete the protease into the fermentation medium. Alternatively, without secretion, the protease can be isolated from the host cell, i.e., purification thereof from the cell mass, for example, by precipitation using ammonium sulfate or ethanol, or by chromatographic purification.

All the above elements can be combined into methods for producing proteases of the invention.

Another subject matter of the invention is an agent that is characterized in that it contains a protease of the invention as described above. Preferably the agent is a detergent or cleaning agent.

Said subject matter of the invention includes all conceivable types of detergents or cleaning agents, both concentrates and also agents to be used in undiluted form, for use on a commercial scale, in the washing machine, or washing or cleaning by hand. They include, for example, detergents for textiles, carpets, or natural fibers for which agents the term detergent is used. They also include, for example, dishwashing agents for dishwashers or manual dishwashing agents or cleaners for hard surfaces such as metal, glass, porcelain, ceramics, tiles, stone, coated surfaces, plastics, wood, or leather for which the term cleaning agent is used, therefore, in addition to manual and automatic dishwashing agents, for example, also scouring agents, glass cleaners, toilet cleaners, etc. The detergents and cleaning agents within the scope of the invention include further washing additives that are added to the actual detergent in manual or automatic textile laundering in order to achieve a further effect. Further, detergents and cleaning agents within the scope of the invention also include textile pre- and post-treatment agents, therefore, agents with which the laundered item is brought into contact before the actual laundering, for example, in order to dissolve stubborn stains, as well as agents that, in a step following the actual textile laundering, impart to the washed item further desirable properties such as a pleasant feel, crease resistance, or low static charge. Fabric softeners, among others, are included among the latter agents.

The washing or cleaning agents of the invention, which may be present as powdered solids, in consolidated particle form, as homogeneous solutions or suspensions, can contain, apart from a protease of the invention, all known ingredients typical in such agents, at least one further ingredient being preferably present in the agent. The agents of the invention can contain in particular surfactants, builders, peroxygen compounds, or bleach activators. They can contain further water-miscible organic solvents, other enzymes, sequestering agents, electrolytes, pH regulators, and/or further aids such as optical brighteners, graying inhibitors, foam regulators, and dyes and fragrances, as well as combinations thereof.

In particular, a combination of a protease of the invention with one or more other ingredients of the agent is advantageous, because an agent of this type in preferred embodiments of the invention has an improved cleaning performance due to the resulting synergisms. Such a synergism can be achieved in particular by the combination of a protease of the invention with a surfactant and/or a builder and/or a peroxygen compound and/or a bleach activator.

Advantageous ingredients of agents of the invention are disclosed in the international patent application WO 2009/121725, beginning therein on page 5, next-to-last paragraph, and ending on page 13 after the second paragraph. Reference is expressly made to this disclosure, and the disclosure content therein is incorporated into the present patent application.

An agent of the invention contains the protease advantageously in an amount of 2 μg to 20 mg, preferably of 5 μg to 17.5 mg, particularly preferably of 20 μg to 15 mg, and very particularly preferably of 50 μg to 10 mg per gram of the agent. Further, the protease, contained in the agent, and/or further ingredients of the agent, can be encased with a substance that is impermeable to the enzyme at room temperature or in the absence of water and becomes permeable to the enzyme under utilization conditions of the agent. Such an embodiment of the invention is thus characterized in that the protease is encased by a substance that is impermeable to the protease at room temperature or in the absence of water. Furthermore, the detergent or cleaning agent itself can also be packaged in a container, preferably an air-permeable container, from which it is released shortly before use or during the washing operation.

In further embodiments of the invention, the agent is characterized in that
(a) it is present in solid form, particularly as a pourable powder with a bulk weight of 300 g/L to 1200 g/L, particularly 500 g/L to 900 g/L, or
(b) it is present in pasty or in liquid form, and/or
(c) it is present as a one-component system, or
(d) it is divided into a plurality of components.

These embodiments of the present invention comprise all solid, powdered, liquid, gel-like, or pasty delivery forms of the agents of the invention, which optionally can consist of multiple phases and be present in compressed or uncompressed form. The agents can be present as a pourable powder, in particular with a bulk weight from 300 g/L to 1200 g/L, in particular 500 g/L to 900 g/L, or 600 g/L to 850 g/L. The solid delivery forms of the agent include further extrudates, granules, tablets, or pouches. Alternatively, the agent can also be liquid, gel-like, or pasty, for example, in the form of a nonaqueous liquid detergent or a nonaqueous paste or in the form of an aqueous liquid detergent or a water-containing paste. Furthermore, the agent can be present as a one-component system. Such agents consist of one phase. Alternatively, an agent can also consist of multiple phases. An agent of this kind is thus distributed into multiple components.

Detergents or cleaning agents of the invention can contain a protease exclusively. Alternatively, they can also contain other hydrolytic enzymes or other enzymes in a concentration appropriate for the effectiveness of the agent. A further embodiment of the invention thus represents agents that moreover comprise one or more further enzymes. All enzymes that can display catalytic activity in the agent of the invention are preferably usable as further enzymes, in particular, a protease, amylase, cellulase, hemicellulase, mannanase, tannase, xylanase, xanthanase, xyloglucanase, β-glucosidase, pectinase, carrageenase, perhydrolase, oxidase, oxidoreductase, or a lipase, as well as mixtures thereof. Further enzymes are contained in the agent advantageously in an amount in each case from 1×10⁻⁸ to 5% by weight, based on active protein. Increasingly preferably, each further enzyme is contained in the agents of the invention, based on active protein, in an amount of 1×10⁻⁷ to 3% by weight, of 0.00001 to 1% by weight, of 0.00005 to 0.5% by weight, of 0.0001 to 0.1% by weight, and particularly preferably of 0.0001 to 0.05% by weight. Particularly preferably, the enzymes exhibit synergistic cleaning performances with regard to specific stains or spots; i.e., the enzymes present in the agent composition are mutually supportive of one another in their cleaning performance. Such a synergism exists very especially preferably between the protease of the invention and another enzyme of an agent of the invention, including particularly between the cited protease and the amylase and/or a lipase and/or a mannanase and/or a cellulase and/or a pectinase. Synergistic effects can occur not only between different enzymes, but also between one or more enzymes and other ingredients of the agent of the invention.

A further subject matter of the invention is a method for cleaning textiles or hard surfaces which is characterized in that an agent of the invention is utilized in at least one process step, or that a protease of the invention is catalytically active in at least one process step, in particular in such a way that the protease is used in an amount of 40 μg to 4 g, preferably of 50 μg to 3 g, particularly preferably of 100 μg to 2 g, and very particularly preferably of 200 μg to 1 g.

This includes both manual and automatic methods, automatic methods being preferred. Methods for cleaning textiles are generally notable in that, in multiple method steps, various substances having cleaning activity are applied to the item to be cleaned and are washed out after the contact period, or that the item to be cleaned is treated in another fashion with a detergent or a solution or a dilution of said agent. The same applies to methods for cleaning all materials other than textiles, particularly hard surfaces. All conceivable washing or cleaning methods can be supplemented, in at least one of the method steps, by the utilization of a detergent or cleaning agent of the invention or a protease of the invention, and then represent embodiments of the present invention. All elements, subject matters, and embodiments described for the proteases of the invention and agents containing them are also applicable to this subject matter of the invention. Reference is therefore expressly made at this juncture to the disclosure at the corresponding point, with the note that this disclosure also applies to the foregoing methods of the invention.

Because proteases of the invention naturally already possess a hydrolytic activity and develop it in media as well that otherwise have no cleaning power, such as, for example, in a simple buffer, an individual and/or the only step of such a method can consist of bringing a protease of the invention, optionally as the only active cleaning component, into contact with the stain, preferably in a buffer solution or in water. This represents another embodiment of this subject matter of the invention.

Alternative embodiments of this subject matter of the invention also represent methods for treating textile raw materials or for textile care, in which a protease of the invention becomes active in at least one process step. Preferred here are methods for textile raw materials, fibers, or textiles having natural constituents, and very particularly for those having wool or silk.

A further subject matter of the invention is the use of an agent of the invention for cleaning textiles or hard surfaces, or a protease of the invention for cleaning textiles or hard surfaces, particularly such that the protease is used in an amount of 40 μg to 4 g, preferably of 50 μg to 3 g, particularly preferably of 100 μg to 2 g, and very particularly preferably of 200 μg to 1 g.

All elements, subject matters, and embodiments described for the proteases of the invention and agents containing them are also applicable to this subject matter of the invention. Reference is therefore expressly made at this juncture to the disclosure at the corresponding point, with the note that this disclosure also applies to for the foregoing use of the invention.

### ILLUSTRATIVE EXAMPLES

All molecular biology procedures follow standard methods, as are specified, for example, in the manual by Fritsch, Sambrook, and Maniatis “Molecular Cloning: A Laboratory Manual,” Cold Spring Harbor Laboratory Press, New York, 1989, or comparable relevant works. Enzymes and kits were used according to the instructions of the particular manufacturer.

### Example 1

### Production of the Proteases

Starting from a protease having an amino acid sequence according to SEQ ID NO:1, a protease variant of the invention was produced by site-directed mutagenesis in the nucleic acid, coding for the protease, by means of the “PHUSION Site-Directed Mutagenesis Kit” (Finnzyme, F541). In so doing, the codons for the indicated amino acid positions were modified so that a substitution of amino acids as specified took place during the translation relative to the amino acid sequence. The protease variants were expressed in a customary manner by the transformation of Bacillus subtilis DB 104 (Kawamura and Doi (1984), J. Bacteriol., Vol. 160 (1), pp. 442-444) with a suitable expression vector and subsequent culturing of the transformands expressing the protease variant. The proteases were purified by ion exchange chromatography from the corresponding cultures.
Protease variant V1 (reference protease): Protease having an amino acid sequence according to SEQ ID NO:1 with the amino acid substitution R99E in the enumeration according to SEQ ID NO:1 (SEQ ID NO:3).
Protease variant V2 (reference protease): Protease having an amino acid sequence according to SEQ ID NO:1 with the amino acid substitutions S3T, V4I, R99E, and V1991 in the enumeration according to SEQ ID NO:1 (SEQ ID NO:4).
Protease variant E1 (protease of the invention): Protease having an amino acid sequence according to SEQ ID NO:1 with the amino acid substitution S3T, V4I, R99E, A188P, and V1991 in the enumeration according to SEQ ID NO:1 (SEQ ID NO:2).

### Example 2

### Stability at a High pH and High Temperature

The proteases V1, V2, and E1 (see Example 1) were tested at 60° C. and pH 10 for temperature stability. The activity was determined at regular intervals by means of the AAPF assay. The half-life was calculated with the assumption of a pseudo 1^{st} order after linearization.

| t½ [min.] | V1 | V2 | E1 |
|---|---|---|---|
| 60° C./pH 10 | 6.5 | 37 | 57 |

It becomes clear that the temperature stability of E1 is also considerably increased compared with the stabilized protease V2.

### Example 3

### Stability in the Presence of Surfactants

The molecules V1, V2, and E1 (see Example 1) were tested for stability at 50° C., pH 8, and in the presence of 1% linear alkylbenzene sulfonate (LAS). The activity was determined at regular intervals by means of the AAPF assay. The half-life was calculated with the assumption of a pseudo 1^{st} order after linearization.

| t½ [sec] | V1 | V2 | E1 |
|---|---|---|---|
| 60° C./pH 10 | 160 | 340 | 470 |

It becomes clear that the temperature stability of E1 is also considerably increased compared with the stabilized protease V2.

### Example 4

### Storage Stability in a Surfactant Matrix

The purified proteases E1, V2, and V1, identical in terms of the active protein, were stored at 37° C. in a detergent matrix and their residual activity after 4 weeks was determined by means of an AAPF measurement.

| Activity [%] | V1 | V2 | E1 |
|---|---|---|---|
| 37° C., 4 weeks | 5% | 35% | 45% |

It is evident that E1 has a considerably improved stability.

### Example 5

### Washing Performance

Proteases V1 and E1, identical in terms of active protein, were used in a miniaturized washing test on stains PC-10, 10N, C-03, EMPA 112, and C-05. To this end, the corresponding stains were incubated in a 48-well microtiter plate in the presence of a detergent matrix and the corresponding protease variant with the same active protein content. In this regard, the lightness of the stains after the washing test was compared relative to a blank without enzyme (delta L). The sum of the delta L values produced the following result:

| Sum delta L | V1 | E1 |
|---|---|---|
| | 37 | 37 |

This shows that E1 does not have a significantly reduced washing performance.

### Example 6

### Cleaning Performance in an Automatic Dishwashing Detergent

The cleaning performance was determined according to the IKW method in a Miele GSL dishwasher at 50° C. (“normal” cycle) and 21° dH ┌German degrees of hardness┐. The results are documented as arithmetic averages. Higher values indicated a better cleaning performance. Formulation F1 was used for rinsing in a dose of 40 g. The composition of F1 can be obtained from the following Table 1; the quantitative data in this case are given in % by weight of active substance, provided the % AS (active substance) is not given along with the trade name.

| | Formulation F1 |
|---|---|
| Potassium tripolyphosphate | 13.75 |
| Phosphonate | 2.40 |
| Acusol 590 38% | 7.50 |
| (Sulfopolymer, AS 38%, Dow Chemical) | |
| Monoethanolamine | 1.35 |
| Acusol 810 18% | 4.95 |
| (Thickener, AS 18%, Dow Chemical) | |
| Sodium carbonate | 5.00 |
| KOH 50% | 7.15 |
| Nonionic surfactant | 1.75 |
| Amylases (based on the amount of active | 0.01 |
| protein) | |
| Proteases (based on the amount of active | 0.11 |
| protein) | |
| Sorbitol | 4.50 |
| Boric acid | 2.00 |
| Calcium chloride | 0.14 |
| Aids (preservative, perfume, glass corrosion | <1 |
| inhibitor, dye, etc.) | |
| Water | To 100 |

Storage stability, at 40° C. after 0, 1, and 4 weeks

| | | Ground | |
|---|---|---|---|
| Product | Week | meat | Starch |
| F1 with protease V2 | 0 | 100 | 100 |
| | 1 | 67 | 82 |
| | 4 | 29 | 39 |
| F1 with protease E1 | 0 | 100 | 100 |
| | 1 | 76 | 94 |
| | 4 | 76 | 79 |

The starting value (week 0) of the cleaning performance according to IKW was used as 100%; the cleaning result after 1 week or 4 weeks of storage at 40° C. in each case refer to the starting value. Protease E1 has a better storage stability than protease V2. Protease E1 has a significantly better cleaning performance after 4 weeks of storage at 40° C. both on the protease-sensitive stain ground meat and the amylase-sensitive stain starch.

1. A protease comprising an amino acid sequence, which has an at least 70% sequence identity with the amino acid sequence set forth in SEQ ID NO:1 over the entire length thereof and which has the amino acid substitutions S3T, V4I, R99D/E, A188P, and V199I based on the numbering according to SEQ ID NO:1.

2. A protease, wherein:
i. the protease is obtainable from a protease according to claim 1 as the parent molecule by a single or multiple conservative amino acid substitution, whereby the protease has the amino acid substitutions S3T, V4I, R99D/E, A188P, and V199I at positions, corresponding to positions 3, 4, 99, 188, and 199 according to SEQ ID NO:1;
ii. the protease is obtainable from a protease according to claim 1 as the parent molecule by fragmentation or by deletion, insertion, or substitution mutagenesis and comprises an amino acid sequence that matches the parent molecule over a length of at least 50, 60, 70, 80, 90, 100, 110, 120, 130, 140, 150, 160, 170, 180, 190, 200, 210, 220, 230, 240, 250, 260, 265, 266, 267, or 268 contiguous amino acids, whereby the protease comprises the amino acid substitutions S3T, V4I, R99D/E, A188P, and V199I at positions, corresponding to positions 3, 4, 99, 188, and 199 according to SEQ ID NO:1;
iii. the protease is obtainable from a protease according to claim 1 as the parent molecule by one or more amino acid substitutions at positions corresponding to positions 36, 42, 47, 56, 61, 69, 87, 96, 101, 102, 104, 114, 118, 120, 130, 139, 141, 142, 154, 157, 193, 205, 211, 224, 229, 236, 237, 242, 243, 255, and 268 of the protease from Bacillus lentus according to SEQ ID NO:1, whereby the protease comprises the amino acid substitutions S3T, V4I, R99D/E, A188P, and V199I at positions, corresponding to positions 3, 4, 99, 188, and 199 according to SEQ ID NO:1.

3. A method for producing a protease comprising the substitution of the amino acids at positions, corresponding to positions 3, 4, 99, 188, and 199 in SEQ ID NO:1, in an original protease, which has an at least 70% sequence identity with the amino acid sequence set forth in SEQ ID NO:1 over the entire length thereof, in such a way that the protease comprises the amino acids 3T, 4I, 99D/E, 188P, and 199I at the corresponding positions.

4. The method according to claim 3, further comprising one or more of the following process steps:
a) introducing a single or multiple conservative amino acid substitution, whereby the protease comprises the amino acid substitutions S3T, V4I, R99D/E, A188P, and V199I at positions, corresponding to positions 3, 4, 99, 188, and 199 according to SEQ ID NO:1;
b) modifying the amino acid sequence by fragmentation or by deletion, insertion, or substitution mutagenesis such that the protease comprises an amino acid sequence, which matches the parent molecule over a length of at least 50, 60, 70, 80, 90, 100, 110, 120, 130, 140, 150, 160, 170, 180, 190, 200, 210, 220, 230, 240, 250, 260, 265, or 266 contiguous amino acids, whereby the protease comprises the amino acid substitutions S3T, V4I, R99D/E, A188P, and V199I at positions, corresponding to positions 3, 4, 99, 188, and 199 according to SEQ ID NO:1;
c) introducing a single or multiple amino acid substitution in one or more of the positions, corresponding to positions 36, 42, 47, 56, 61, 69, 87, 96, 101, 102, 104, 114, 118, 120, 130, 139, 141, 142, 154, 157, 193, 205, 211, 224, 229, 236, 237, 242, 243, 255, and 268 of the protease from Bacillus lentus according to SEQ ID NO:1, whereby the protease comprises the amino acid substitutions S3T, V4I, R99D/E, A188P, and V199I at positions, corresponding to positions 3, 4, 99, 188, and 199 according to SEQ ID NO:1.

5. A nucleic acid coding for a protease according to claim 1.

6. A vector containing a nucleic acid according to claim 5 selected from the group consisting of a cloning vector and expression vector.

7. A non-human host cell that contains a nucleic acid according to claim 5 wherein the non-human host cell secretes the protease into the medium surrounding the non-human host cell.

8. A method for producing a protease comprising
a) culturing a host cell according to claim 7; and
b) isolating the protease from the culture medium or from the host cell.

9. An agent, comprising at least one protease according to claim 1 in an amount of 2 μg to 20 mg per gram of agent,
or comprising at least one protease according to either claim 1 and the protease in the agent is encased by a substance that is impermeable to the protease at room temperature or in the absence of water.

10. A method for cleaning textiles or hard surfaces, wherein an agent according to claim 9 is utilized in at least one process step such that the protease is used in an amount of 40 μg to 4 g per use.
